(19) United States
(12) Patent Application Publication
(10) Pub. No.: US 2013/0331946 A1
(43) Pub. Date: Dec. 12, 2013

(54) EXPANDABLE JOINT IMPLANT

(71) Applicant: Ortho-Space Ltd., Caesarea (IL)

(72) Inventor: Shaul Shohat, Kfar HaOranim (IL)

(73) Assignee: Ortho-Space Ltd., Caesarea (IL)

(21) Appl. No.: 13/967,973

(22) Filed: Aug. 15, 2013

Related U.S. Application Data
(63) Continuation of application No. 13/147,688, filed on Aug. 3, 2011.
(60) Provisional application No. 61/202,211, filed on Feb. 6, 2009.

(51) Int. Cl. A61F 2/42

(52) U.S. Cl. CPC A61F 2/4241
USPC 623/21.11

(57) ABSTRACT

An expandable implantable device sized for occupying space in a cavity formed between small bones in a human hand or foot. Comprising a first smooth surface on which a first small bone may slide. May include an opening extending through the device for promoting fibrotic development through the opening from a direction from both thumb metacarpal and trapezium. An inflation cannula and/or needle may be attached to inflation port and inflation valve for introducing expansion fluid into the device. In some embodiments, an implant is inserted in a deflated mode and positioned so that a thumb metacarpal abuts distal side of the device when expanded, and trapezium abuts a proximal side thereof. At least one of distal side and proximal side include a smooth surface for allowing relative movement of thumb metacarpal or/and trapezium with respect to the device.

### RELATED APPLICATIONS

This application is a continuation of U.S. patent application Ser. No. 13/147,688 filed on 3 Aug. 2011, which is a national phase filing under 35 U.S.C. 371 of International Application No. PCT/IB2010/050562 filed on 8 Feb. 2010, which claims the benefit of U.S. Provisional Application No. 61/202,211 filed on 6 Feb. 2009. The contents of the preceding stated documents are incorporated by reference as if fully set forth herein.

### FIELD AND BACKGROUND OF THE INVENTION

The present invention, in some embodiments thereof, relates to body implantable devices and, more particularly, but not exclusively, to a small bones implant device and a method for implanting between small bones.

A human thumb metacarpal is connected to the wrist through a first carpometacarpal (CMC) joint also known as the trapeziometacarpal joint. A base of the thumb metacarpal articulates with a saddle shaped trapezium, the saddle shaped articulation providing a stability required for grasping actions. Furthermore, the saddle shaped articulation allows for thumb motion in a tri-axial mode, which may make the joint relatively susceptible to arthritic degeneration. Soft tissue surrounding the joint, including the beak ligament, may additionally contribute to the arthritic degeneration by causing joint surface subluxation. Damage to the CMC joint may therefore be debilitating and may require surgical intervention when other measures do not suffice.

Patients suffering from arthritis of the CMC joint may recur to surgical intervention as a possible solution to their problem. A commonly employed solution is CMC joint arthroplasty also referred to as trapezectomy. This may be performed by an open surgical procedure or by an arthroscopic procedure, a goal of the surgical intervention being to obtain a painless strong thumb without losing motion or causing deformation.

A number of reconstructive procedures are known in the art for treating CMC joint arthritis. These include interpositional arthroplasty, resection arthroplasty of the trapezium, resection interpositional arthroplasty of the trapezium, total and partial joint replacement arthroplasty of the CMC joint (several types of prosthetic joints), and arthroscopic procedures such as CMC arthroplasty and CMC joint arthrodesis.

U.S. Pat. No. 7,037,342 “IMPLANT FOR RECONSTRUCTION OF JOINTS” relates to “A spacer member (1) is intended to be placed between the ends of the bones which are to be connected, one end of the spacer member being designed to form a joint surface against one of the bone ends (6,7). A joint-stabilizing connection (2,3) is arranged to connect the bones. The spacer member (1) is made of at least one tissue-compatible material.”

US Publication No. 2006/0241778 “INTERPOSITIONAL BIARTICULAR DISK” relates to “An interpositional biarticular disk implant (11) having a circular peripheral rim, a generally toroidal axial center opening (13) and convex upper and lower surfaces (15, 17) is implanted between resected concave surfaces of the metacarpal base and the trapezium or other carpal bone in a CMC joint replacement. The disk (11) is anchored in operative position through the use of a flexable cond, such as a harvested tendon that passes through the center opening (13) and through osseous passageways created in the two facing bones.”

### SUMMARY OF THE INVENTION

There is provided in accordance with an exemplary embodiment of the invention, a soft, expandable, implantable device sized for spacing between small bones comprising a first smooth surface on which a first small bone may slide.

In an exemplary embodiment of the invention, said device is biodegradable in the body. Optionally or alternatively, said small bones comprise bones of a human hand. Optionally or alternatively, said small bones comprise bones of a human foot.

In an exemplary embodiment of the invention, said small bones comprise at least one of a trapezium, a trapezoid bone, a metacarpal bone, and a scaphoid bone.

In an exemplary embodiment of the invention, the device comprises an inflation port for inflating said device.

In an exemplary embodiment of the invention, the device comprises at least one passage extending from a distal side to a proximal side of the device for promoting fibrotic development between said small bones.

In an exemplary embodiment of the invention, the device comprises at least one passage extending from a first lateral side to an opposite second lateral side of the device for promoting fibrotic development between said small bones. Optionally or alternatively, at least one of said at least one passage includes has a diameter that is at least 20% of a maximal extent of the device.

In an exemplary embodiment of the invention, the device comprises a mesh for promoting fibrotic development between said small bones. Optionally, the mesh covers a portion of an external surface of said device. Optionally, the mesh is covered by a biodegradable material.

In an exemplary embodiment of the invention, the mesh is included inside said device.

In an exemplary embodiment of the invention, a portion of the surface of said device is coated with a fibrosis promoting substance.

In an exemplary embodiment of the invention, an inflation liquid used to expand said device includes a bio-active material which is eluted though a wall of said implant. Optionally, the material comprises a fibrosis promoting substance.

In an exemplary embodiment of the invention, the device is configured to cushion between the small bones.

In an exemplary embodiment of the invention, the device comprises an annular shaped portion.

In an exemplary embodiment of the invention, the device comprises a cylindrical shaped portion.

In an exemplary embodiment of the invention, said first smooth surface is located on a proximal side of said device.

In an exemplary embodiment of the invention, said first small bone is a thumb metacarpal bone.

In an exemplary embodiment of the invention, the device comprises a distal side with a second smoothed surface on which a second small bone may slide. Optionally, said second small bone is a trapezium.

In an exemplary embodiment of the invention, the device comprises an anti-adhesive applied to the smooth surface on the proximal side and/or on the distal side to prevent bone adhesion.

In an exemplary embodiment of the invention, the device comprises a biodegradable material including PLA, PLGA, caprolactone, polycaprolactone, polydiaxone, or any combination thereof.

In an exemplary embodiment of the invention, said device is designed to rupture within a time period of 1 to 30 weeks following expansion. Optionally, said device is designed to rupture within a time period of 6 to 10 weeks following expansion.

In an exemplary embodiment of the invention, said device is designed to completely biodegrade within a time period of 3-30 months following expansion. Optionally, said device is designed to completely biodegrade within a time period of 6-12 months following expansion.

In an exemplary embodiment of the invention, the device comprises an anchor adapted for attaching said device to an articular capsule.

In an exemplary embodiment of the invention, the device comprises an anchor adapted for attaching said device to a bone.

In an exemplary embodiment of the invention, the device comprises a sleeve incorporating at least one of a seal and a valve.

In an exemplary embodiment of the invention, the device comprises said sizing comprises a diameter within a range of a factor of 0.8 to 1.2 of a diameter of said small bone adjacent said device.

In an exemplary embodiment of the invention, said sizing comprises a thickness of within the range of a factor of 0.5 to 2 of a natural distance between said small bones.

There is therefore provided in accordance with an exemplary embodiment of the invention, a method for implanting between small bones comprising:
inserting an expandable device between at least two small bones; and expanding the device.

In an exemplary embodiment of the invention, said small bones comprise bones of a human hand and/or a wrist.

In an exemplary embodiment of the invention, said small bones comprise bones of a human foot.

In an exemplary embodiment of the invention, the method comprises inserting the device through a small perforation in an articular capsule.

In an exemplary embodiment of the invention, the method comprises removing bone tissue from part of the at least two small bones to prepare a cavity between the bones. Optionally, the method comprises positioning the device inside the cavity.

In an exemplary embodiment of the invention, expanding the device comprises inflating the device with a liquid, a gas, a gel, or any combination thereof.

In an exemplary embodiment of the invention, the method comprises moving the at least two small bones relative to the device after a period of at least 3 days following implantation. Optionally, moving the at least two small bones comprises sliding the bones along surfaces of the device.

In an exemplary embodiment of the invention, the method comprises allowing the device to biodegrade. Optionally, the method comprises allowing the device to rupture within a time period of 6 to 10 weeks following implantation.

In an exemplary embodiment of the invention, the method comprises allowing the device to 100% biodegrade within a time period of 6-18 months following implantation.

In an exemplary embodiment of the invention, the method comprises promoting fibrotic development between the at least two small bones.

In an exemplary embodiment of the invention, the method comprises covering a portion of a surface of the device with a fibrosis promoting substance.

In an exemplary embodiment of the invention, the method comprises preventing the at least two small bones from adhering to the device.

In an exemplary embodiment of the invention, the method comprises promoting fibrotic development by covering a portion of the device with a mesh.

In an exemplary embodiment of the invention, the method comprises performing an arthroscopic surgical intervention for inserting the device between the at least two small bones.

In an exemplary embodiment of the invention, the method comprises performing a Trapezectomy for inserting the device between the at least two bones. Optionally, the Trapezectomy is a Partial Trapezectomy.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A and 1B schematically illustrate an exemplary view of the bones in the hand and an enlarged view of an area of the hand showing the thumb metacarpal and the trapezium, respectively;

FIGS. 2A-2E schematically illustrate steps involved in replacing a CMC joint in a hand using an exemplary expandable joint implant device, according to an embodiment of the present invention;

FIGS. 3A-3G schematically illustrate different configurations of the exemplary joint implant device, according to some embodiments of the present invention;

FIGS. 4A and 4B schematically illustrate different configurations of the exemplary joint implant device including anchoring means, according to some embodiments of the present invention;

FIG. 5 schematically illustrates another configuration of the exemplary joint implant device, according to some embodiments of the present invention;

FIG. 6 schematically illustrates a flow chart of a method for performing a CMC joint implant including the exemplary joint implant device; and

FIG. 7 schematically illustrates several views A-D of an exemplary joint implant device, according to some embodiments of the present invention.

### DESCRIPTION OF EMBODIMENTS OF THE INVENTION

The present invention, in some embodiments thereof, relates to body implantable devices and, more particularly, but not exclusively, to a small bones implant device and a method for implanting between small bones.

An aspect of some embodiments of the present invention relates to an expandable implantable device configured to be implanted in between small bones, for inducing deposition of tissues between the bones. Throughout this disclosure, the “expandable implantable device” may be used interchangeably with “expandable joint implant device”, “expandable cushioning device”, “expandable spacer”, “joint implant device”, “expandable device”, “joint device”, “implant”, and “device”. The device may be used for partially replacing a CMC joint in a hand. Optionally, the device is used for wholly replacing the CMC joint in the hand. In some exemplary embodiments, the joint implant device may be used at other articular sites (joint areas) such as, for example, between phalanges of a hand and/or a foot, at the metatarsalphalangeal joint, between the scaphoid and the trapezoid, and at other small joints in the foot and/or the hand.

According to some embodiments, the implant is configured to substantially conform to bony surfaces in the joint area and to serve as a cushion between the bones. Optionally, the implant includes a size which matches a width of the wider bone. Optionally, the size matches a width of the narrower bone. Optionally, the implant includes a size which ranges from 50%-90% of the width of the wider bone. Additionally or alternatively, the size ranges from 50%-90% of the width of the narrower bone. Optionally, the implant allows relative movement of the bones while providing for tissue repair and stabilization. The implant is further configured to serve as a spacer for maintaining a separation between the bones. Optionally, length and configuration are restored to the joint substantially reducing anatomical deformation, for example, a thumb length and shape may be maintained. An advantage of using the implant over other devices known in the art is that patient rehabilitation may be initiated soon following surgical intervention, for example, within a period ranging from 1-14 days. Optionally, patient rehabilitation may start within a period of 1-3 days, 3-7 days, 7-10 days.

In some embodiments, the joint implant device may include an inflatable device such as for example, a balloon, which is inserted in a deflated state into a cavity in the joint area following a partial or complete resection of a joint surface. Once in position, the balloon may be inflated to an expanded state with a liquid, a gas, or a gel, introduced into the balloon through an inflation port. Optionally, the inflation port is located inside the balloon when in an expanded state so as to substantially prevent injury or irritation from rubbing against edges of the port. Optionally, inflation is done by temporarily attaching an inflation cannula or inflation needle to the inflation port and removing the cannula or needle when the balloon is expanded to a required size. Additionally or alternatively, the inflation port is biodegradable. Optionally, the inflation port includes a one-way valve which may be biodegradable.

In some embodiments of the present invention, an expanded size of the device may be, for example, 22 mm x 20 mm x 12 mm. Optionally, an expanded size may be 20 mm x 12 mm x 8 mm. Additionally or alternatively, the expanded size of the device may be such that a length of the device does not exceed 30 mm, a width does not exceed 30 mm, and a height does not exceed 20 mm. An expanded shape of the device may include any polyhedral shape, such as for example, rectangular, trapezoidal, cylindrical, octagonal. Optionally, the expanded shape may include a spherical shape or any curved variations of a spherical shape such as, for example, ellipsoid. A non-expanded size of the device may not exceed a length of 30 mm, a width of 30 mm, and a height of not more than 10 mm (for example, 6 mm, 4 mm, 3 mm or less). A wall thickness of the device may range from less than 1 mm to 5 mm, for example 1 mm, 2 mm, 3 mm. A modulus of elasticity of the device may be equivalent to that of a cartilage or a tendon. Optionally, the modulus of elasticity is in a range of 30%-150% of that of a cartilage, or a tendon. Inserting the balloon in a deflated state allows for surgical intervention involving joint arthroscopy or arthroscopic CMC joint arthroplasty, the balloon inserted through a relatively small perforation in the articular capsule. Optionally, the surgical intervention may include open surgery. Additionally or alternatively, the surgical intervention may include a full Trapezectomy or a partial Trapezectomy. Optionally, the implant may include a sponge-like device or other type of device which may be compressed for insertion and expands once positioned inside the cavity.

In some embodiments, the device may include a biodegradable material such as, for example, PLA, PLGA, polycaprolactone, polydiaxone, or other biocompatible biodegradable material, or any combination thereof, suitable for lasting a period of time in a range from 1 week to 1 year, optionally from 1 month to 4 months, optionally from 6 weeks to 12 weeks, for example 8 weeks. Additionally or alternatively, the device may include a biodegradable material such that the device ruptures within a period of 5-15 weeks from implantation, for example 7-8 weeks, the ruptured device biodegrading within a period of time ranging from 6-18 months from implantation, for example 9-14 months. Optionally, the device may include a non-biodegradable, biocompatible material such as, for example, polyethylene, polyurethane, silicon, other polymeric or non-polymeric biocompatible materials, or any combination thereof. Optionally, the device may be seamless thereby allowing an improved homogeneity in shape and/or degradation, improved structural durability to inner and/or outer stresses. More details about exemplary degradable materials and/or balloons, and ways of producing thereof, are described in US Publication No. 2008/0033471 titled “DEVICE SYSTEM AND METHOD FOR TISSUE DISPLACEMENT OR SEPARATION”, the disclosure of which is fully incorporated herein by reference.

In some embodiments, the device may include a cylindrical shape and may include one or more openings (passages) extending from a first side of the device to a second opposing side of the device, for allowing fibrotic development (fibrosis) between the bones from both sides of the device (from the side of the trapezoid and the side of the metacarpal). Optionally, the passages may angularly extend from the first side to the second side. Optionally, the passages are positioned and oriented so as to achieve a desired pattern of fibrotic growth. Optionally, the passages may cover an area ranging from 10%-50% of the total area of the first side and the second side. Creating fibrotic bridges between the bones, additionally to filling the joint area and strengthening the surrounding articular capsule, may serve to permit a full range of motion and to prevent bone shortening, for example, thumb shortening. Optionally, the device may include an internal cylindrical shape forming two concentric rings (one rings inside the other). Optionally, the external ring biodegrades before the internal ring. Optionally, the device may be star-shaped so that radial extensions may assist in promoting fibrosis. Optionally, the device may include any other shape suitable for promoting fibrosis while allowing the device to serve as a cushion and/or spacer. Optionally, an exterior of the device may be partially or wholly covered with a mesh for promoting fibrosis, in some cases the mesh covering those areas in contact with bone and which do not interfere with movement of the bones. Optionally, such a mesh is distributed to achieve a desired pattern of fibrotic growth. Optionally, such a mesh may be covered by a biodegradable material so as to not interfere with movement of the bones while the capsule is being strengthened and only then being exposed to promote fibrosis between the bones. Additionally or alternatively, an interior of the device may include such a mesh, and may protrude through a surface of the device as the device biodegrades. Optionally, the surface of the device may be coated with a slow-release substance that promotes fibrosis such as, for example, FGF. Optionally, walls of passages are coated with the substance which promotes fibrotic development. Additionally or alternatively, walls of passages are coated with a substance which substantially inhibits fibrotic development. Optionally, the surface may be coated with an anti-inflammatory substance such as, for example, steroids and or antibiotics. Optionally, the device may include seams to promote fibrosis. Optionally, the device may be filled with a fibrosis promoting agent that may be dispersed into the surrounding when the device ruptures and/or at least partially degrades.

In some embodiments, the opposing first and second sides of the device may include at least one smooth surface for allowing movement of the bones relative to the device, including sliding along the surface. For example, the first (thumb) metacarpal may move along the surface of the first side (distal side) of the device, and the device may move with respect to the trapezium along the surface of its opposing side (proximal side). Optionally, the surface may be coated with a substance which substantially prevents bone adherence. Optionally, such a substance enhances sliding of a bone on the surface. Optionally, the surface may include a substance which substantially prevents sliding of a bone over the surface.

In some embodiments of the present invention, the device may include a shape resembling a “dumbbell” including two expanded ends joined together by a connecting portion so that movement in the joint area is transmitted to each end of the device while substantially maintaining movement between the bones and the surfaces of the device to a minimum (reducing wear on the bones and possible pain). In this case, at least one of the surfaces in contact with a bone is coarse or rough thereby minimizing a relative movement of the surface with respect to the bone and allowing a transfer of movement to the connecting portion.

In some embodiments of the present invention, the device may include anchoring means (anchor) for substantially preventing dislodgement of the device during movements within the articulation. The anchor may be located on a palmar side of the device and may be attached to the articulate capsule. Optionally, the anchor may be positioned on the device at an angle not less than 30 degrees from the inflation port. Optionally, the anchor may be arrow-shaped and is configured to pierce into the palmar capsule where it may be embedded. Optionally, the anchor may include a hook for securing the attachment to the capsule or to bone. Optionally, the anchor may be adhered to the bone or to the articular capsule by means of an adhesive. Optionally, the adhesive is biodegradable. Additionally or alternatively, the anchor may be inflatable and is inflated together with the rest of the device via the inflation port. Optionally, the anchor includes a separate inflation port and is inflated following attachment to the capsule (or the bone). Optionally, a same inflation port includes separate conduits for inflating the device and the anchor. Optionally, the anchor is an integral extension of the device and is of a same material as the device. Optionally, the anchor is a separate extension of the device, and may or may not, be of a same material as the device.

An aspect of some embodiments of the present invention relates to a method for implanting an expandable device in between at least two small bones using minimal invasive surgery such as, for example, arthroscopic intervention. In an exemplary embodiment, the method includes perforating a small hole in an articular capsule; removing bone tissue from part of the at least two small bones to form a cavity; inserting the device in a non-expanded state into the cavity and expanding the device to a size where the device may serve both as a cushion and a spacer in between the at least two bones; and starting patient rehabilitation after a period ranging from 1-14 days. Optionally, patient rehabilitation may start within a period of 1-3 days, 3-7 days, 7-10 days.

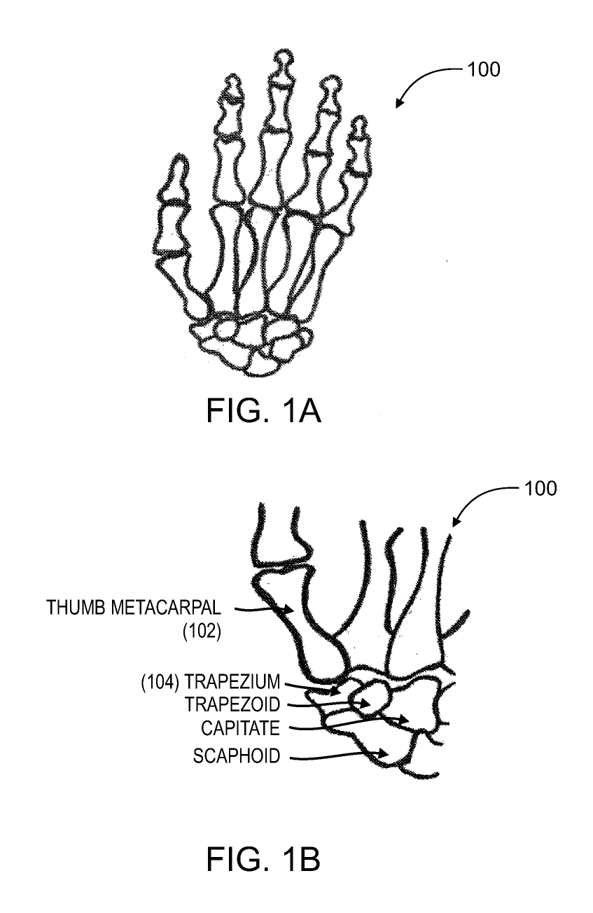

For purposes of better understanding some embodiments of the present invention, reference is first made to FIGS. 1A and 1B which schematically illustrate an exemplary view of the bones in the hand 100 and an enlarged view of an area of the hand showing the thumb metacarpal 102 and the trapezium 104, respectively.

Before explaining at least one embodiment of the invention in detail, it is to be understood that the invention is not necessarily limited in its application to the details of construction and the arrangement of the components and/or methods set forth in the following description and/or illustrated in the drawings. The invention is capable of other embodiments or of being practiced or carried out in various ways.

Before explaining at least one embodiment of the invention in detail, it is to be understood that the invention is not necessarily limited in its application to the details set forth in the following description. The invention is capable of other embodiments or of being practiced or carried out in various ways.

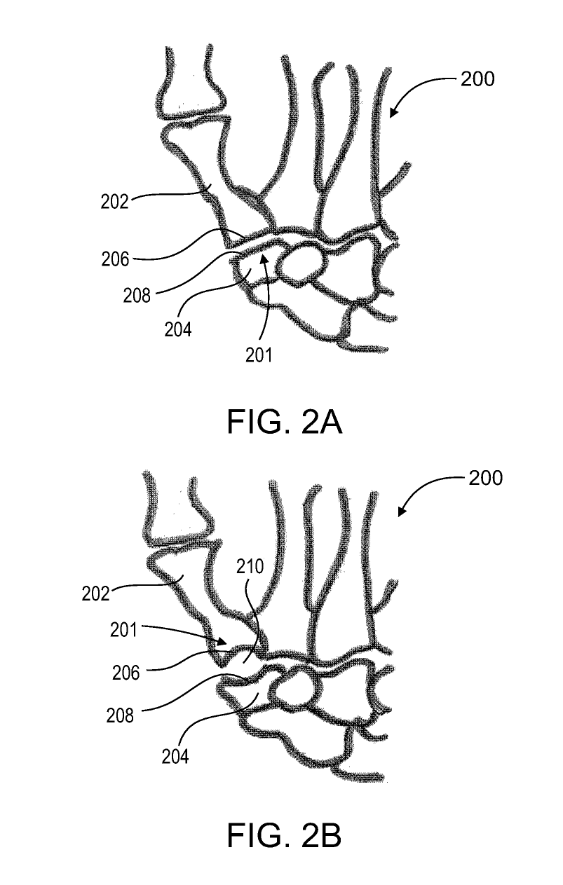

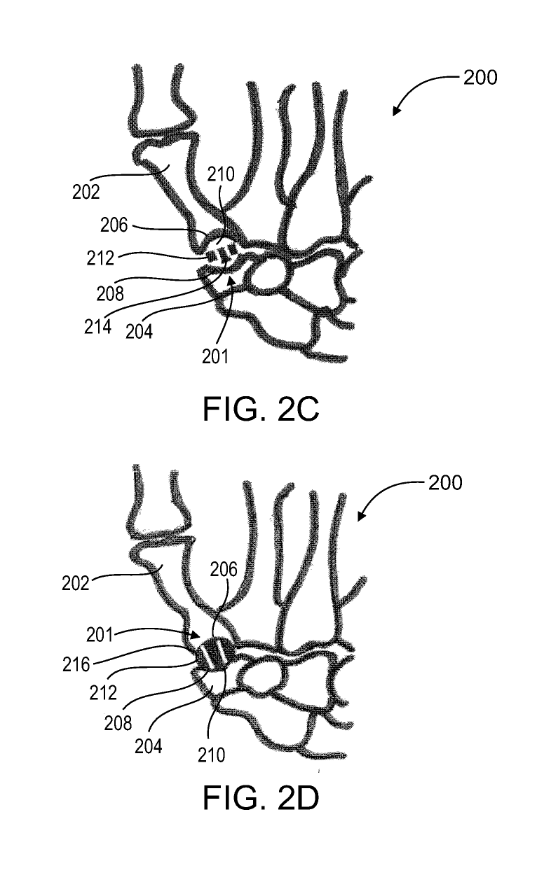

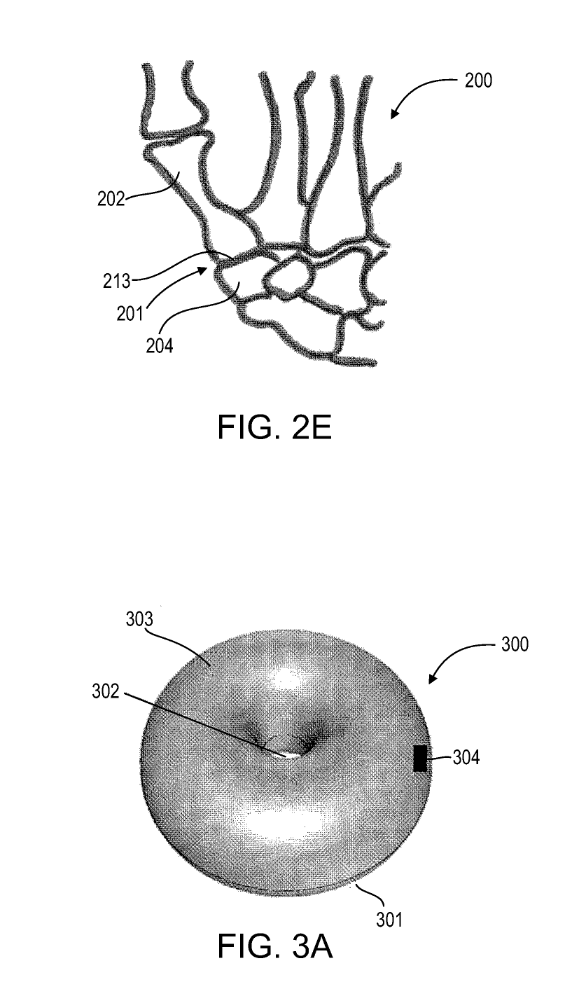

Referring now to the drawings, FIGS. 2A-2E schematically illustrate steps included in treating a CMC joint in a hand 200 using an exemplary expandable joint spacer implant device 212, according to an embodiment of the present invention. Optionally, the treated joint is in another articular site, for example, elsewhere in hand 200 or in a foot (not shown).

FIG. 2A shows a portion of base 206 of thumb metacarpal 202 and a portion of inferior surface 208 of trapezium 204 cut so as to facilitate access to a joint area 201.

FIG. 2B shows a cavity 210 formed in joint area 201 by cutting portions of base 206 and inferior surface 208, the cavity configured to receive implant 212.

FIG. 2C shows implant 212 in a non-expanded state and positioned inside cavity 210 in joint area 201, between base 206 and inferior surface 208. Optionally, implant 212 is in a compressed state or in a deflated state.

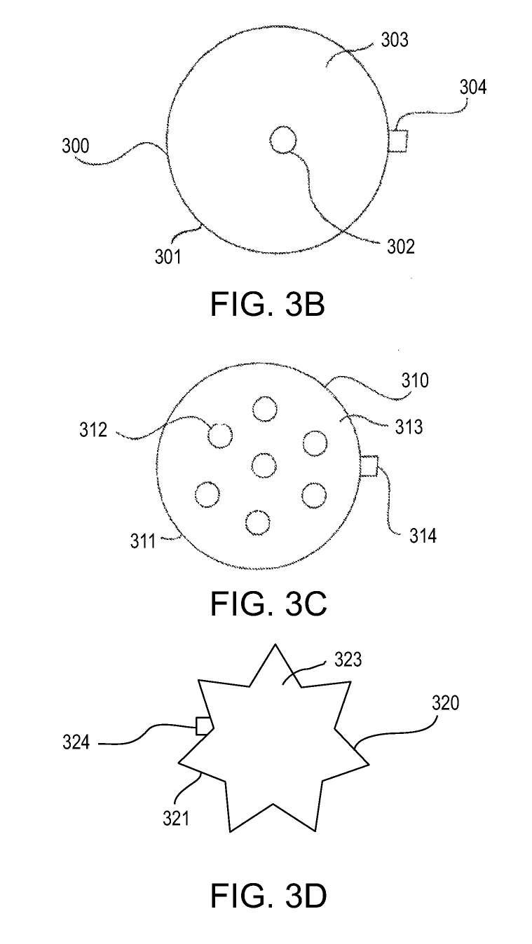

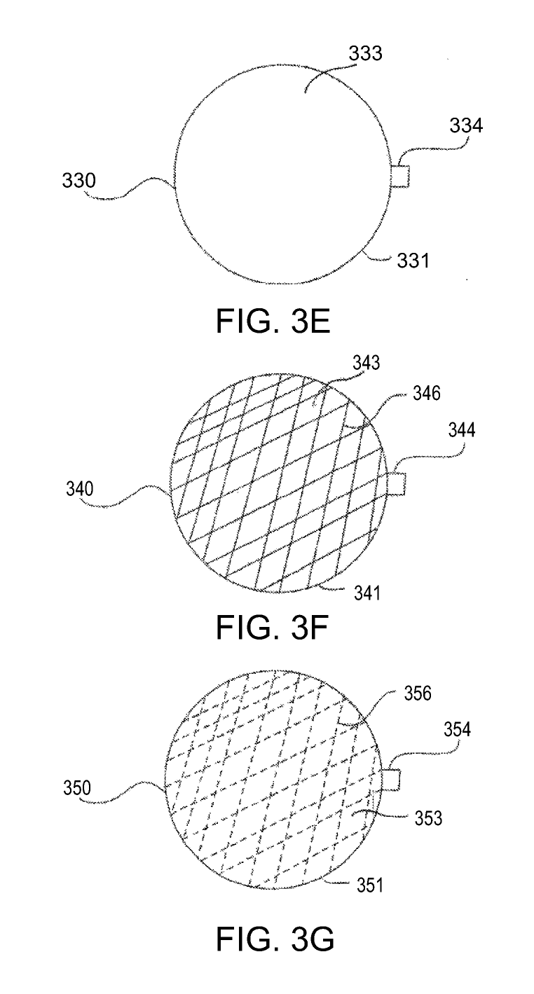

Implant 212 may include a balloon or similar inflatable device; a sponge-like device; or any other expandable device suitable to be inserted in a compressed state, or a deflated state into cavity 210 through a relatively small perforation in the articular capsule. Implant 212 may include a biocompatible and/or a biodegradable material. Biocompatible materials may include, for example, polyethylene, polyurethane, silicon, or other biocompatible polymeric or non-polymeric materials, or any combination thereof. Biodegradable materials may include, for example, PLA, PLGA, polycaprolactone, polydiaxone or other biodegradable material, or any combination thereof, suitable for degrading within a period of any predetermined window of time, optionally between 6-12 weeks from insertion into cavity 210, for example, optionally within 8 weeks. Optionally, device 212 may be similar to those devices shown in FIGS. 3A-5 described further on below.

Implant 212 may include an inflation port 214 through which an expansion fluid, which may be a liquid, a gas, or a gel, may be introduced to inflate the device. The expansion fluid may be a biocompatible fluid, and/or a biodegradable fluid. For example, the liquid may be a 0.9% saline, a Ringer solution or a Hartman solution. The gel may be, for example, an absorbable haemostatic agent such as gelatin, cellulose, or bovine collagen, or a biodegradable synthetic adhesive such as poly ethylene glycol (PEG). The gas may be for example, oxygen, nitrogen, or any other gas readily absorbed by the human body, or any combination thereof.

FIG. 2D shows implant 212 is in an expanded (inflated) state, following introducing of the expansion fluid into the device through inflation port 214. Inflation port 214 is not visible as the port is inside device 212 following inflation of the device.

Inflation of implant 212 is done by temporarily attaching an inflation cannula, optionally an inflation needle, to inflation port 214, and injecting the expansion fluid into the device. Once device 212 is expanded to the appropriated size, inflation port 214 is sealed and the inflation cannula removed.

Once expanded to the appropriate size, device 212 optionally fills cavity 210 and optionally conforms to the shape of the cavity, including the reshaped portions of base 206 and inferior surface 208. In the expanded state, implant 212 serves as a cushion and/or a spacer between thumb metacarpal 202 and trapezium 204. Optionally, device 212 includes holes for promoting fibrotic development between thumb metacarpal 202 and trapezium 204. Optionally, a contour of device 212 is suitable for promoting fibrotic development, for example by including extensions such as radial extensions in a starred shape. Additionally or alternatively, device 212 may include a mesh for promoting fibrotic development or may be coated with a fibrosis promoting substance such as, for example, FGF. Optionally, device 212 may include a coating of an anti-inflammatory substance such as, for example, steroids and/or antibiotics. Optionally, device 212 may include a seam, which may optionally assist to promote fibrosis. Optionally, device 212 may be seamless.

In the expanded state, device 212 may be configured to allow thumb metacarpal 202 to slide along a distal side of the device abutting with base 206, and for the device to slide relative to trapezium 204 along a proximal side of the device abutting inferior surface 208. Optionally, device 212 slides relative to thumb metacarpal 202 and/or to trapezium 204.

Device 212 may optionally include an anchor 216 for substantially preventing possible dislodgement of the device from within cavity 210 when expanded. Anchor 216 may be attached to the articular capsule or to a bone, for example, to the scaphoid below the trapezium or to the trapezoid next to the trapezium.

FIG. 2E shows joint area 201 following biodegradation of implant 212 and a fibrotic bridge 213 formed between thumb metacarpal and trapezium 204 (synfibrosis). Fibrotic bridge 213 filled joint area 201 and strengthened the surrounding articular capsule. Due to an optional continuous relative movement of metacarpal 202 and trapezium 204 therebetween and with respect to implant 212, a complete bone fusion is substantially prevented and a synfibrosis, a synarthrosis or a synostosis is optionally formed in the space previously occupied by implant 212, allowing at least partial movement of thumb metacarpal 202 with respect to trapezium 204.

Reference is now also made to FIGS. 3A-3G which schematically illustrate different, non-limiting, configurations of an exemplary joint implant device, according to some embodiments of the present invention. The implants shown at 300, 310, 320, 330, 340, and 350 may be similar to that shown in FIGS. 2C and 2D at 212. The embodiments shown are not intended to be limiting in any form, and it should be evident to an ordinary person skilled in the art that many other configurations (including device shapes, hole shapes, extensions, etc.) may be used.

FIGS. 3A and 3B show a perspective view of implant 300 and a layout view of the implant. Implant 300 includes a torus (optionally cylindrical) shape with an opening 302 (passage) extending through the device (“donut-shaped”) for promoting fibrotic development through the opening from a direction from both thumb metacarpal 202 and trapezium 204. Optionally, opening 302 which is shown with a circular shape may include other shapes, for example rectangular, triangular, star-shaped, an 8-shape, or other polygonal shapes. Optionally, proximal side 301 and distal side 303 may include a coating of a slow-release substance for promoting fibrosis, and/or with an anti-inflammatory substance such as, for example, a steroid or an antibiotic. Optionally, proximal side 301 and/or distal side 303 may be treated with a substance to prevent bone adhesion. Additionally or alternatively, the substance may enhance bone movement along the surfaces. Implant 300 is inserted into cavity 210 in a deflated mode and is positioned so that thumb metacarpal 202 abuts distal side 303 of the device when expanded and trapezium 204 abuts proximal side 301. Both distal side 303 and proximal side 301 include a smooth surface for allowing relative movement of thumb metacarpal 202 and trapezium 204 with respect to device 300. Implant 300 includes an inflation port 304 to which an inflation cannula or needle may be attached for introducing expansion fluid into the device.

FIG. 3C shows a layout view of implant 310. Implant 310 includes a cylindrical shape with a plurality of openings 312 (passages) extending through the device for promoting fibrotic development through the openings from a direction from both thumb metacarpal 202 and trapezium 204. Optionally, opening 312 which is shown with a circular shape may include other shapes, for example rectangular, triangular, star-shaped, an 8-shape, or other polygonal shape, or any combination thereof. Optionally, proximal side 311 and distal side 313 may include a coating of a slow-release substance for promoting fibrosis, and/or with an anti-inflammatory substance such as, for example, a steroid or an antibiotic. Optionally, proximal side 311 and/or distal side 313 may be treated with a substance to prevent bone adhesion. Additionally or alternatively, the substance may enhance bone movement along the surfaces Implant 310 is inserted into cavity 210 in a deflated mode and is positioned so that thumb metacarpal 202 abuts distal side 313 of the device when expanded and trapezium 204 abuts proximal side 311. Both distal side 313 and proximal side 311 include a smooth surface for allowing relative movement of thumb metacarpal 202 and trapezium 204 with respect to device 310. Implant 310 includes an inflation port 314 to which an inflation cannula or needle may be attached for introducing expansion fluid into the device.

FIG. 3D shows a layout view of implant 320. Implant 320 includes a starred shape including radial extensions for promoting fibrotic development through the extensions from both thumb metacarpal 202 and trapezium 204. Optionally, proximal side 321 and distal side 323 may include a coating of a slow-release substance for promoting fibrosis, and/or with an anti-inflammatory substance such as, for example, a steroid or an antibiotic. Optionally, proximal side 321 and/or distal side 323 may be treated with a substance to prevent bone adhesion. Additionally or alternatively, the substance may enhance bone movement along the surfaces. Optionally, implant 320 may include one or more passages extending directly or angularly from proximal side 321 to distal side 323. Implant 320 is inserted into cavity 210 in a deflated mode and is positioned so that thumb metacarpal 202 abuts distal side 323 of the device when expanded and trapezium 204 abuts proximal side 321. Both distal side 323 and proximal side 321 include a smooth surface for allowing relative movement of thumb metacarpal 202 and trapezium 204 with respect to device 320. Implant 320 includes an inflation port 324 to which an inflation cannula or needle may be attached for introducing expansion fluid into the device.

FIG. 3E shows a layout view of implant 330 which includes a cylindrical shape. Implant 330 is inserted into cavity 210 in a deflated mode and is positioned so that thumb metacarpal 202 abuts a distal side 333 of the device when expanded and trapezium 204 abuts a proximal side 331. Both distal side 333 and proximal side 331 include a smooth surface for allowing relative movement of thumb metacarpal 202 and trapezium 204 with respect to device 330. Optionally, proximal side 331 and distal side 331 may include a coating of a slow-release substance for promoting fibrosis, and/or with an anti-inflammatory substance such as, for example, a steroid or an antibiotic. Optionally, proximal side 331 and/or distal side 333 may be treated with a substance to prevent bone adhesion. Additionally or alternatively, the substance may enhance bone movement along the surfaces. Implant 330 includes an inflation port 334 to which an inflation cannula or needle may be attached for introducing expansion fluid into the device.

FIG. 3F shows a layout view of implant 340. Implant 340 includes a mesh 346 covering a portion, optionally a whole, of a proximal side 341 and/or a distal side 343 of the implant for promoting fibrotic development from both thumb metacarpal 202 and trapezium 204. Optionally, only portions of proximal side 341 and/or distal side 343 not in contact with moving bones are covered by the mesh. Additionally or alternatively, mesh 346 is covered by a biodegradable material which exposes the mesh only after a period of time during which the articular capsule is strengthened, for promoting fibrosis between thumb metacarpal 202 and trapezium 204. The material provides both distal side 343 and proximal side 341 with a smooth surface for allowing relative movement of thumb metacarpal 202 and trapezium 204 with respect to device 340. Optionally, the material prevents bone adhesion. Optionally, proximal side 341 and distal side 343 may include a coating of a slow-release substance for promoting fibrosis, and/or with an anti-inflammatory substance such as, for example, a steroid or an antibiotic. Implant 340 is inserted into cavity 210 in a deflated mode and is positioned so that thumb metacarpal 202 abuts distal side 343 of the device when expanded and trapezium 204 abuts proximal side 341. Implant 340 includes an inflation port 344 to which an inflation cannula or needle may be attached for introducing expansion fluid into the device.

FIG. 3G shows a layout view of implant device 350 including a mesh 356 inside the device for promoting fibrotic development from both thumb metacarpal 202 and trapezium 204. Device 350 may be similar to device 340 with the exception that mesh 356 is internally located in the expandable portion of the device, and protrudes through a surface of the device as the device biodegrades. Optionally, mesh 356 is exposed once a major portion of device 350, or optionally a whole of the device, biodegrades.

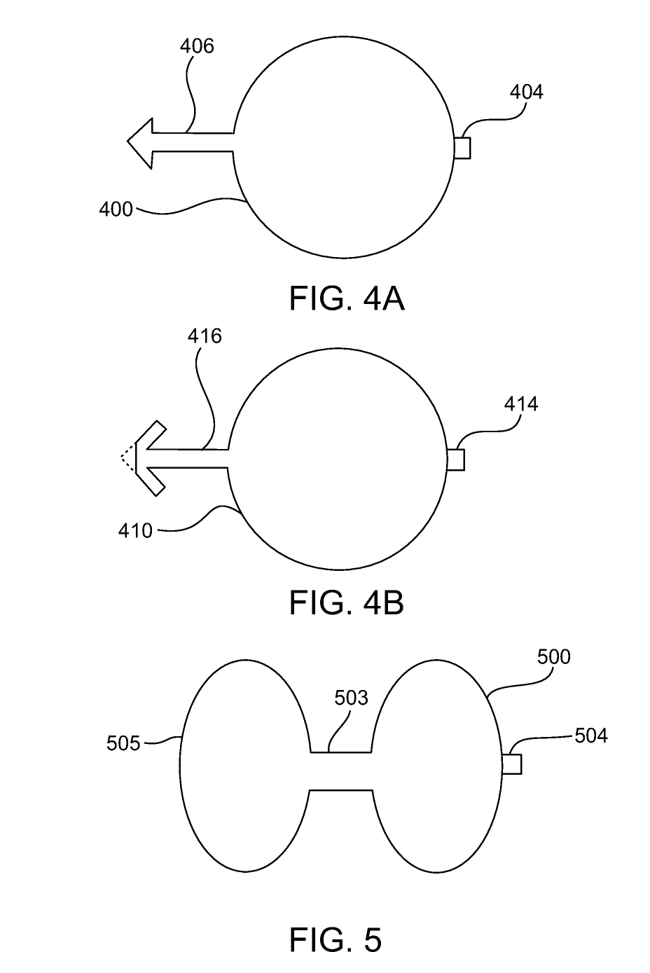

Reference is now also made to FIGS. 4A and 4B which schematically illustrate different, non-limiting configurations of the exemplary joint implant device including anchoring means, according to some embodiments of the present invention. The implants, shown at 400 and 410, may be similar to that shown in FIGS. 2C and 2D at 212, or FIGS. 3A-3G at 300, 310, 320, 330, 340, or 350, respectively, with a difference that implants 400 and 410 include an anchor 406 and 416 respectively. The embodiments shown are not intended to be limiting in any form, and it should be evident to an ordinary person skilled in the art that many other configurations (including device shapes, hole shapes, extensions, etc.) may be used.

Anchor 406 in FIG. 4A and anchor 416 in FIG. 4B are configured to substantially prevent dislodgement of device 400 and 410 respectively, during movements within cavity 210. Anchors 406 and 416 may be located on a palmar side of device 400 and 410, respectively, and may be attached to the articulate capsule. Optionally, as shown in FIG. 4A, anchor 406 may be arrow-shaped and is configured to pierce into the palmar capsule where it may be embedded. Optionally, as shown in FIG. 4B, anchor 416 may include a hook for securing the attachment to the capsule or to bone. Optionally, anchor 416 may include an arrow-shaped head to facilitate piercing of the capsule prior to attachment of the hooks. Additionally or alternatively, anchor 406 and/or 416 is attached to the articular capsule or the bone by means of a biodegradable adhesive.

According to some embodiments, anchors 406 and/or 416 may be inflatable and may be inflated together with the expansive portion (inflatable portion) of device 400 and 410 via inflation ports 404 and 414, respectively. Optionally, anchors 406 and/or 416 may include separate inflation ports and may be inflated following attachment to the capsule (or the bone). Optionally, inflation ports 404 and/or 414 include separate conduits for inflating the device and the anchor. Optionally, anchors 406 and/or 416 may be an integral extension of device 400 and/or 410, respectively, and of a same material as the device. Optionally, 406 and/or 416 may be a separate extension of device 400 and/or 410, respectively, and may or may not be of a same material as the device. Optionally, anchors 406 and/or 416 are made of a biocompatible and/or biodegradable material.

Reference is now also made to FIG. 5 which schematically illustrates another configuration of an exemplary joint implant device 500, according to some embodiments of the present invention. Implant 500 may be similar to that shown in FIGS. 2C and 2D at 212, or FIGS. 3A-3G at 300, 310, 320, 330, 340, or 350, respectively, or FIGS. 4A or 4B at 400 or 410, respectively with a difference that implant 500 includes a “dumbbell” shape.

Implant 500, when expanded, includes two relatively large expanded end sections 505 joined together by a relatively narrower connecting portion 503. Inflation of end sections 505 and connecting portion 503 is by means of an expansion fluid introduced through inflation port 504, as previously described for other embodiments. Optionally, connecting portion 503 is a non-inflatable element of a suitable size to be inserted into cavity 210 with end sections 505 deflated. Implant 500 is configured to substantially restrain movement of thumb metacarpal 202 and trapezium 204 relative to the implant. Movement between the two bones is through connecting portion 503, which acts as a joint, with movement of each bone transmitted to end sections 505 which act as supports.

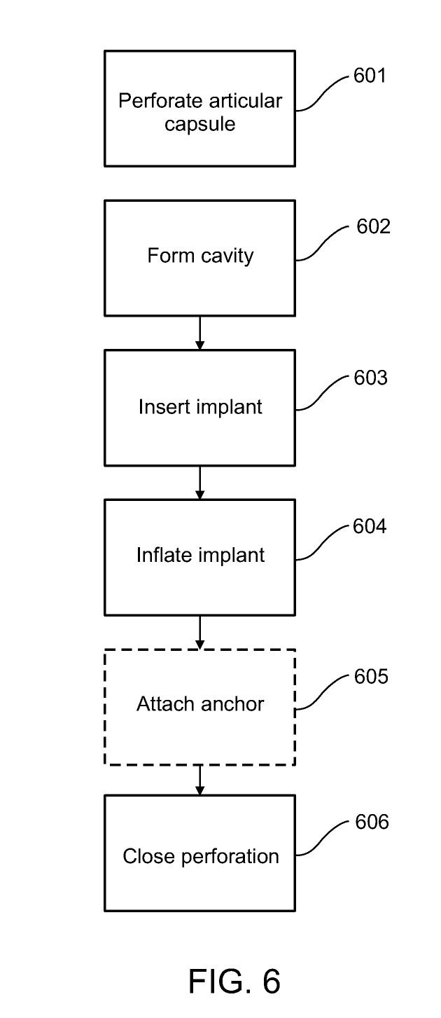

Reference is now made to FIG. 6 which schematically illustrates a flow chart of a method for performing a CMC joint implant including exemplary joint implant device 212. Optionally, any of the devices 300, 310, 320, 330, 340, 350, 400, 410, or 500 may be used for implementing the method. The method described below is not intended to be limiting in any way and it should be evident to an ordinary person skilled in the art that there may be many other ways of implementing the method, including, for example, using different steps, a different order of the steps, skipping steps, inserting steps.

Optionally at 601, a partial trapezectomy is to be performed using arthroscopy. A hole is perforated in the articular capsule and a portion of base 206 in thumb metacarpal 202 is cut. A portion of inferior surface 208 in trapezium 204 is also cut, both cuts to facilitate access to joint area 201 to prepare cavity 210. Optionally, a full trapezectomy may be performed wherein trapezium 204 is completely removed. Optionally, the partial trapezectomy may be removed using open surgery.

Optionally at 602, base 206 and inferior surface 208 are further cut and shaped to form cavity 210 in joint area 201. Cavity 210 is of a size to allow non-expanded device 212 to be inserted into the cavity and positioned such that, when expanded, will act as a cushion and/or spacer between thumb metacarpal 202 and trapezium 204. Optionally, expanded device 212 is secured within cavity 210 any may not be dislodged through the hole in the articular cavity. Cavity 210 shall also allow for device 212 to be positioned such that there is relatively easy access to inflation port 214 for expanding the device and for sealing the inflation port once the device is expanded.

Optionally at 603, implant 212 is inserted into cavity 210. An inflation cannula which may include an inflation needle for connecting to inflation port 214 may be configured to clasp device 212 at a distal end, for positioning the device inside cavity 210. Optionally, device 212 may be rolled on a distal end of the inflation cannula in a deflated state for inserting in cavity 210. Optionally, other methods known in the art for placing implants using arthroscopy may be used.

Optionally at 604, an expansion fluid is injected through the inflation cannula into inflation port 214. The expansion fluid may include a liquid, a gas, or a gel, or any combination thereof. Device 212 is expanded to cover a portion, optionally a whole, of cavity 210 forming a cushion and/or spacer between thumb metacarpal 202 and trapezium 204. Device 212 partially, optionally wholly, conforms to a shape of the bone surfaces of base 206 and inferior surface 208. Once expanded to the desired length, inflation port 214 may be sealed. Optionally, inflation cannula may be detached from device 212 and extracted from the articular capsule. Optionally, the inflation cannula is not extracted for possible use in attaching anchor 216 and/or for performing other arthroscopic operations.

Optionally at 605, anchor 216 may be attached to the articulate capsule. Optionally, anchor 216 may be attached to a bone, for example, to the scaphoid located below the trapezium or to the trapezoid located next to the trapezium.

Optionally at 606, the perforation in the articular capsule is closed and the arthroscopic procedure is finalized. Patient may start rehabilitation after a period ranging from 1-14 days. Optionally, patient rehabilitation may start within a period of 1-3 days, 3-7 days, 7-10 days. Optionally, device 212 may include a biodegradable material such that the device ruptures within a period of 5-15 weeks from implantation, for example 7-8 weeks. During this time the articular capsule is strengthened and limited fibrotic development occurs around and through the device. Optionally, the ruptured device biodegrades within a period of time ranging from 6-18 months from implantation, for example 9-14 months, during which time substantially complete fibrotic development occurs.

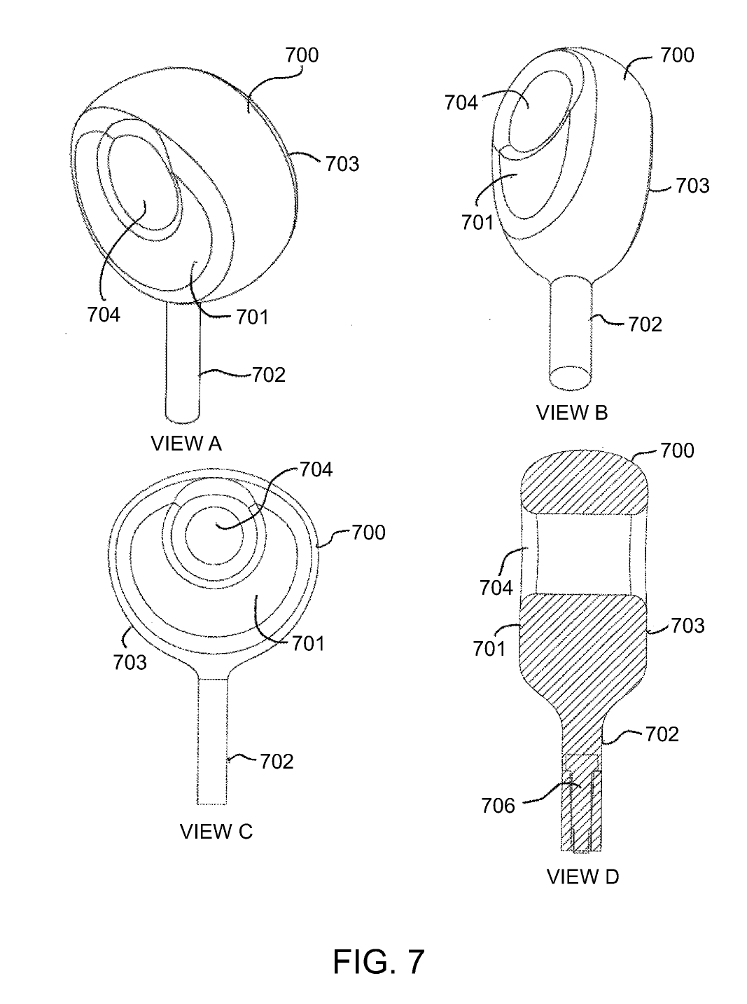

Reference is now also made to FIG. 7 which schematically illustrates several views A-D of an exemplary joint implant device 700, according to some embodiments of the present invention. Implant 700 may be similar to that shown in FIGS. 2C and 2D at 212, or FIGS. 3A-3G at 300, 310, 320, 330, 340, or 350, respectively, or FIGS. 4A or 4B at 400 or 410, respectively. The exemplary embodiment shown herein is not intended to be limiting in any form, and it should be evident to an ordinary person skilled in the art that many other configurations (including device shapes, hole (passage) shapes, number of passages, etc.) may be used for inplant 700.

Views A and B show a perspective view of implant 700, view C shows a layout view of the implant, and view D shows a sectional view of the implant, according to an exemplary embodiment. Implant 700 which may include a shape as shown includes an opening 704 (passage) extending through the device for promoting fibrotic development through the opening from a direction from both thumb metacarpal 202 and trapezium 204. Optionally, passage 704 which is shown with a circular shape may include other shapes, for example rectangular, triangular, star-shaped, an 8-shape, or other polygonal shapes. Passage 704 is optionally distally positioned from a one-way inflation valve 706 included inside an inflation port 702 substantially limiting possible damage to the device due to the insertion pressure of an expansion fluid. An inflation cannula and/or needle may be attached to inflation port 702 and inflation valve 706 for introducing the expansion fluid into the device. Optionally, inflation port 702 includes a biodegradable material. Optionally, inflation valve includes a biodegradable material.

According to some embodiments, proximal side 701 and distal side 703 may include a coating of a slow-release substance for promoting fibrosis, and/or with an anti-inflammatory substance such as, for example, a steroid or an antibiotic. Optionally, proximal side 701 and/or distal side 703 may be treated with a substance to prevent bone adhesion. Additionally or alternatively, the substance may enhance bone movement along the surfaces. Implant 700 is inserted into cavity 210 in a deflated mode and is positioned so that thumb metacarpal 202 abuts distal side 703 of the device when expanded and trapezium 704 abuts proximal side 701. Both distal side 703 and proximal side 701 include a smooth surface for allowing relative movement of thumb metacarpal 202 and trapezium 204 with respect to device 700.

The terms “comprises”, “comprising”, “includes”, “including”, “having” and their conjugates mean “including but not limited to”. This term encompasses the terms “consisting of and “consisting essentially of”.

The phrase “consisting essentially of means that the composition or method may include additional ingredients and/or steps, but only if the additional ingredients and/or steps do not materially alter the basic and novel characteristics of the claimed composition or method.

As used herein, the singular form “a”, “an” and “the” include plural references unless the context clearly dictates otherwise. For example, the term “a compound” or “at least one compound” may include a plurality of compounds, including mixtures thereof.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

What is claimed is:

1. A method for implanting between small bones in a human hand comprising:
forming a cavity between at least two of the small bones in the hand by cutting a portion of at least one of thumb metacarpal and trapezium;
inserting into said cavity an expandable implantable device comprising a first smooth surface on which a first small bone may slide; and
expanding said device to occupy a space between said at least two small bones, such that said device acts as a cushion between said at least two small bones, to permit full range of thumb motion and to prevent thumb shortening.

2. The method of claim 1, wherein said forming comprises removing bone tissue from part of said at least two small bones.

3. The method of claim 1, wherein said inserting is facilitated through a small perforation in articular capsule in the hand, such that said device when expanded is secured within said cavity without dislodging through said perforation.

4. The method of claim 1, comprising inserting said device in a deflated mode and positioning said device so that when expanded said thumb metacarpal abuts distal side of said device and said trapezium abuts proximal side of said device.

5. The method of claim 1, comprising performing an arthroscopic surgical intervention for inserting said device between said at least two small bones.

6. The method of claim 1, comprising performing a trapezectomy for inserting said device between said at least two bones.

7. The method of claim 6, wherein said trapezectomy is a partial trapezectomy.

8. The method of claim 1, wherein said expanding comprises inflating said device with a liquid, a gas, a gel, or any combination thereof.

9. The method of claim 8, wherein said inflating includes injecting said liquid, gas, gel, or any combination thereof, through an inflation cannula into an inflation port of said device, and wherein the method further comprises sealing said inflation port, detaching said inflation cannula from said device, and extracting said inflation cannula from said cavity.

10. The method of claim 1, wherein said device includes an anchor, and further comprising attaching said anchor to an articular capsule or to one of said at least two small bones in the hand.

11. The method of claim 1, further comprising promoting fibrotic development between said at least two small bones.

12. The method of claim 11, wherein said fibrotic development is effected by covering a portion of a surface of said device with a fibrosis promoting substance.

13. The method of claim 11, wherein said fibrotic development is effected by said device including a mesh.

14. The method of claim 11, wherein said fibrotic development is effected by said device including at least one hole.

15. The method of claim 11, wherein said fibrotic development is effected by a contour of said device.

16. The method of claim 15, wherein said contour comprises extensions of said device.

17. The method of claim 1, comprising moving said at least two small bones relative to said device while providing for tissue repair and stabilization.

18. The method of claim 17, wherein said moving is performed after a period of at least 3 days following implantation.

19. The method of claim 17, wherein said moving comprises sliding said at least two small bones along surfaces of said device.

20. The method of claim 1, further comprising allowing said device to rupture within a time period of 6 to 10 weeks following implantation.

21. The method of claim 1, further comprising allowing said device to biodegrade within a time period of 6-18 months following implantation.

22. The method of claim 1, comprising configuring and sizing said device to have a diameter within a range of a factor of 0.8 to 1.2 of a diameter of a small bone adjacent said device.

23. The method of claim 1, comprising configuring and sizing said device to have a thickness of within a range of a factor of 0.5 to 2 of a natural distance between said at least two small bones.

24. The method of claim 1, comprising configuring and sizing said device to match a width of a wider bone of said at least two small bones or to include a size ranging from 50% to 90% of said width of said wider bone.

25. The method of claim 1, comprising configuring and sizing said device to match a width of a narrower bone of said at least two small bones or to include a size ranging from 50% to 90% of said width of said narrower bone.
